(19) United States
(12) Patent Application Publication
(10) Pub. No.: US 2014/0039316 A1
(43) Pub. Date: Feb. 6, 2014

(54) ULTRASONIC DIAGNOSTIC APPARATUS AND ULTRASONIC IMAGE PROCESSING METHOD

(71) Applicants: Toshiba Medical Systems Corporation, Otawara-shi (JP); Kabushiki Kaisha Toshiba, Minato-ku (JP)

(72) Inventors: Kenichi ICHIOKA, Nasushiobara-shi (JP); Takuya SASAKI, Nasu-gun (JP)

(73) Assignees: Toshiba Medical Systems Corporation, Otawara-shi (JP); Kabushiki Kaisha Toshiba, Minato-ku (JP)

(21) Appl. No.: 14/041,848

(22) Filed: Sep. 30, 2013

Related U.S. Application Data
(63) Continuation-in-part of application No. PCT/JP2013/067562, filed on Jun. 26, 2013.

(30) Foreign Application Priority Data
Jun. 29, 2012 (JP) 2012-148016
Jun. 24, 2013 (JP) 2013-131455

(51) Int. Cl. A61B 8/13; A61B 8/08

(52) U.S. Cl. CPC A61B 8/13; A61B 8/0841
USPC 600/439; 600/443

(57) ABSTRACT

According to one embodiment, an insertion area setting unit sets an insertion area in a predetermined range with a planned insertion route of a puncture needle in volume data being a central axis. An expansion image generation unit generates an expansion image expressing a brightness distribution on a side surface of the insertion area in the volume data by two-dimensional polar coordinates defined by a rotational angle around the central axis and a distance from a reference point on the central axis. A display unit displays the expansion image.

### CROSS REFERENCE TO RELATED APPLICATIONS

This application is a Continuation application of PCT Application No. PCT/JP2013/067562, filed Jun. 26, 2013 and based upon and claiming the benefit of priority from Japanese Patent Applications No. 2012-148016, filed Jun. 29, 2012; and No. 2013-131455, filed Jun. 24, 2013, the entire contents of all of which are incorporated herein by reference.

### FIELD

Embodiments described herein relate generally to an ultrasonic diagnostic apparatus and an ultrasonic image processing method.

### BACKGROUND

An ultrasonic diagnostic apparatus radiates ultrasonic pulses from transducers built in an ultrasonic probe into the body of a patient. The ultrasonic diagnostic apparatus receives reflected ultrasonic waves generated by differences in acoustic impedance of living tissues via transducers. The ultrasonic diagnostic apparatus acquires various types of biological information based on the reception signals generated by the reception of reflected ultrasonic waves. A recent ultrasonic diagnostic apparatus can electronically control the transmission/reception direction and focal point of ultrasonic waves by controlling the delay times of driving signals supplied to a plurality of transducers or reception signals obtained from the transducers. Using such an ultrasonic diagnostic apparatus allows the operator to easily observe an image in real time by the simple operation of bringing the distal end portion of an ultrasonic probe into contact with the body surface. Ultrasonic diagnostic apparatuses are widely used for morphological and functional diagnoses of organs in living bodies.

Recently, there has been developed a method of performing a predetermined examination or medical treatment by inserting a puncture needle into a lesion in a patient (examination/medical treatment target region) under the observation of the image obtained by using an ultrasonic diagnostic apparatus. For example, this apparatus displays the two-dimensional image acquired from a slice including the puncture needle. The two-dimensional image depicts a lesion and a puncture needle. The operator inserts the puncture needle into the lesion while observing the lesion and the puncture needle and grasping their positional relationship.

A puncture guideline is superimposed on a two-dimensional image to support the accurate insertion of the puncture needle. A puncture guideline is a linear mark indicating a planned puncture route of the puncture needle. A puncture guideline is generated based on, for example, information from a puncture adapter attached to an ultrasonic probe.

It is premised that a puncture needle is linearly inserted into the body of a patient. However, a general puncture needle does not have sufficient hardness. For this reason, if the elasticity (hardness) characteristics of living tissues in a puncture route are not uniform, the operator may insert the puncture needle in a direction different from the planned puncture route indicated by a puncture guideline. If the puncture needle deviates from a two-dimensional image slice, it is impossible to grasp the distal end portion of the puncture needle on the two-dimensional image.

In order to solve this problem, the following ultrasonic diagnostic apparatus is proposed. This ultrasonic diagnostic apparatus acquires volume data in a three-dimensional region in the body of a patient including a lesion by using a two-dimensional array ultrasonic probe including a two-dimensional array of a plurality of transducers and detects the position information of the distal end of the puncture needle inserted into the three-dimensional region. This ultrasonic diagnostic apparatus generates a plurality of slice images perpendicular to each other with reference to the distal end portion of the puncture needle based on volume data, and displays these slice images. The operator can accurately grasp the distal end portion of the puncture needle by observing these slice images even when the puncture needle is inserted in a bent state.

The above method using volume data allows to accurately grasp the position information of the distal end portion of a puncture needle even if the actual puncture route of the puncture needle deviates from a planned puncture route due to the nonuniformity of the elasticity characteristics of living tissues.

However, the region observed by the above display method is limited to MPR slices perpendicular to each other which are set with reference to the distal end portion of the puncture needle. It is difficult to efficiently observe morphological information in a wide range with reference to the distal end portion of the puncture needle before or during insertion.

### BRIEF DESCRIPTION OF THE DRAWINGS

FIG. 1 is a block diagram showing the arrangement of an ultrasonic diagnostic apparatus according to an embodiment.

FIG. 2 is a block diagram showing the arrangements of a transmission/reception unit and signal processor in FIG. 1.

FIG. 3A is a view for explaining the relationship between an ultrasonic probe in FIG. 1 and an ultrasonic transmission/reception direction, showing the positional relationship between an ultrasonic probe 2 and a pqr orthogonal coordinate system.

FIG. 3B is a view for explaining the relationship between the ultrasonic probe in FIG. 1 and an ultrasonic transmission/reception direction, with a transmission/reception direction Op of ultrasonic waves being projected on a pr plane in the pqr coordinate system in FIG. 3A.

FIG. 3C is a view for explaining the relationship between the ultrasonic probe in FIG. 1 and an ultrasonic transmission/reception direction, with a transmission/reception direction θq of ultrasonic waves being projected on a qr plane in the pqr coordinate system in FIG. 3A.

FIG. 4 is a block diagram showing the arrangement of the volume data generation unit of the ultrasonic diagnostic apparatus according to this embodiment.

FIG. 5 is a block diagram showing the arrangement of a position information calculation unit in FIG. 1.

FIG. 6 is a view showing the insertion area set by an insertion area setting unit in FIG. 1.

FIG. 7 is a block diagram showing the arrangement of an expansion image generating unit in FIG. 1.

FIG. 8 shows the expansion image generated by the expansion image generation unit in FIG. 1.

FIG. 9 is a view showing the expansion image on which a luminal region is superimposed and which is generated by the expansion image generation unit in FIG. 1.

FIG. 10 shows an example of the puncture support image generated by the puncture support image generation unit in FIG. 1 and including a distance mark.

FIG. 11 shows an example of the puncture support image generated by the puncture support image generation unit in FIG. 1 and including a distance mark corresponding to zones through which the puncture needle has already passed and a distance mark corresponding to zones through which the puncture needle has not yet passed.

FIG. 12 shows an example of the puncture support image generated by the puncture support image generation unit in FIG. 1 and including an intersecting position mark.

FIG. 13 is a flowchart showing a typical example of puncture support image generation/display processing performed under the control of a system controller in FIG. 1.

FIG. 14A is a view showing a puncture support image according to Application Example 1 and explaining a puncture support image concerning zones [q2 to q5].

FIG. 14B is a view showing a puncture support image according to Application Example 1 and explaining a puncture support image concerning zones [q0 to q3].

FIG. 15 shows a puncture support image according to a modification of Application Example 1 and a puncture support image including the expansion image on which a puncture target region is superimposed.

FIG. 16A is a view showing the ultrasonic probe to which probe marks according to this embodiment are attached when viewed from the front.

FIG. 16B is a view showing the ultrasonic probe to which the probe marks according to this embodiment are attached when viewed from above.

FIG. 17 is a view showing the puncture support image generated by the puncture support image generation unit in FIG. 1 and including azimuth marks.

### DETAILED DESCRIPTION

An embodiment of this disclosure will be described below with reference to the accompanying drawings.

In general, according to one embodiment, an ultrasonic diagnostic apparatus includes an ultrasonic probe, a transmission unit, a reception unit, a volume data generation unit, a setting unit, an expansion image generation unit, and a display unit. The ultrasonic probe includes transducers. The transmission unit transmits ultrasonic waves to a scanning target region in a subject via the transducers. The reception unit receives ultrasonic waves from the scanning target region via the transducers. The volume data generation unit generates volume data concerning the scanning target region based on a reception signal from the reception unit. The setting unit sets a predetermined range in the volume data to a region of interest. The predetermined range has a central axis coinciding with a planned insertion route of a puncture needle. The expansion image generation unit generates an expansion image expressing a brightness distribution on a side surface of the region of interest in the volume data by two-dimensional polar coordinates defined by a rotational angle around the central axis and a distance from a reference point on the central axis. The display unit displays the expansion image.

The ultrasonic diagnostic apparatus according to this embodiment is used for a puncturing operation. A puncture needle according to this embodiment may be a puncture needle for biopsy (living tissue examination) aimed at harvesting a lesion tissue. This puncture needle may also be a cautery treatment puncture needle such as an RFA puncture needle which can perform a cautery treatment for a lesion. For a practical description of this embodiment, assume that the puncture needle according to the embodiment is a puncture needle for biopsy.

The ultrasonic diagnostic apparatus according to this embodiment has no limitation on the type of ultrasonic probe to be used as long as it can generate volume data. That is, the ultrasonic probe according to the embodiment may be a two-dimensional array type probe including a plurality of transducers arrayed two-dimensionally or a one-dimensional array type probe including a plurality of transducers arrayed one-dimensionally. When using a two-dimensional array type probe, the ultrasonic diagnostic apparatus acquires volume data by ultrasonically scanning a three-dimensional region via a plurality of transducers arrayed two-dimensionally. When using one-dimensional array type probe, the ultrasonic diagnostic apparatus acquires volume data by repeatedly ultrasonically scanning a scanning plane via a one-dimensional transducer array while mechanically moving it.

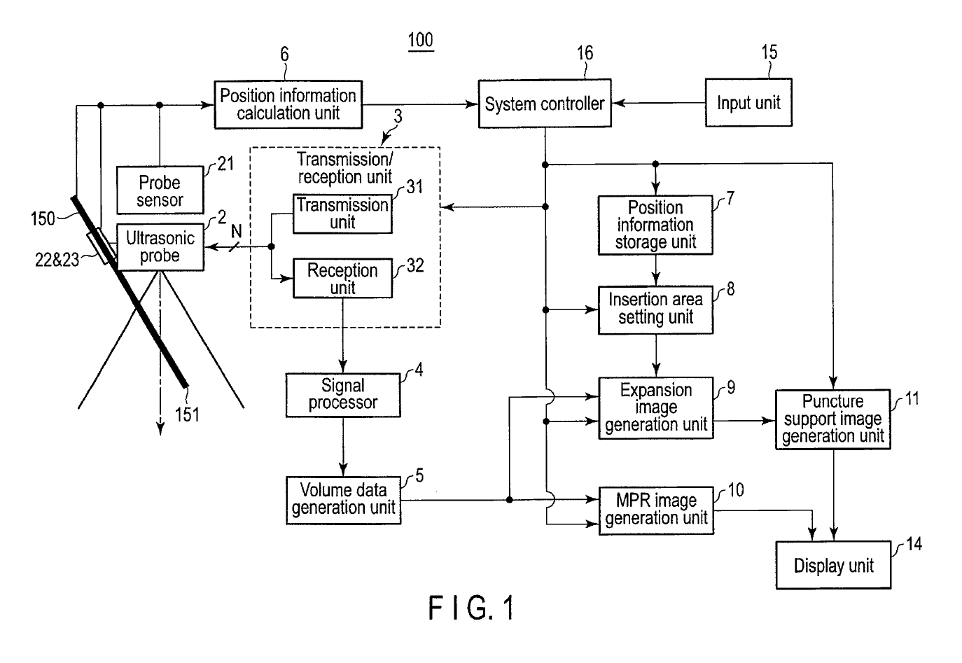

FIG. 1 is a block diagram showing the overall arrangement of an ultrasonic diagnostic apparatus 100 according to this embodiment. As shown in FIG. 1, the ultrasonic diagnostic apparatus 100 includes an ultrasonic probe 2, a transmission/reception unit 3, a signal processor 4, a volume data generation unit 5, a position information calculation unit 6, and a position information storage unit 7.

The ultrasonic probe 2 includes a plurality of transducers. The plurality of transducers radiate ultrasonic waves (ultrasonic pulses) to a three-dimensional scanning region in the body of a patient before or during the insertion of a puncture needle 150. The plurality of transducers convert ultrasonic waves (reflected ultrasonic waves) from the scanning region into electrical reception signals. The ultrasonic probe 2 incorporates or includes, as a peripheral unit, probe sensors 21 for grasping the position and direction of the ultrasonic probe 2 in a real space. The probe sensor 21 is a position sensor provided for the ultrasonic probe 2. Each probe sensor 21 detects the position of the ultrasonic probe 2. A wall surface of the ultrasonic probe 2 is provided with a puncture adapter 22 and an adapter sensor 23. The puncture adapter 22 defines the initial insertion position of the puncture needle 150 used for examination or medical treatment on a lesion and holds the puncture needle 150 so as to allow it to slide in the inserting direction. The adapter sensor 23 is a position sensor provided for the puncture adapter 22. The adapter sensor 23 detects the distal end position of the puncture adapter 22. The distal end position of the puncture adapter 22 corresponds to the initial insertion position of the puncture needle 150. The distal end of the puncture needle 150 is provided with a puncture needle sensor 151. The puncture needle sensor 151 detects the distal end position of the puncture needle 150.

The transmission/reception unit 3 supplies driving signals to the plurality of transducers to radiate ultrasonic waves to a scanning region. The transmission/reception unit 3 performs phasing addition of reception signals obtained from these transducers via a plurality of channels. The signal processor 4 generates B-mode data by processing the reception signal after the phasing addition. The volume data generation unit 5 generates volume data based on the above B-mode data obtained for each transmission/reception direction of ultrasonic waves.

The position information calculation unit 6 calculates the position information of the distal end of the puncture needle 150 based on position signals from the puncture needle sensor 151, the probe sensors 21, and the adapter sensor 23. The position information of the distal end of the puncture needle 150 will be referred to as needlepoint position information hereinafter. Needlepoint position information is the position information of the distal end of the puncture needle 150 relative to the ultrasonic probe 2. The position information calculation unit 6 calculates the initial position information of the distal end of the puncture needle 150 based on position signals from the puncture needle sensor 151, the probe sensor 21, and the adapter sensor 23. The initial position information of the distal end of the puncture needle 150 will be referred to as initial needlepoint position information hereafter. Initial needlepoint position information is the position information of the distal end of the puncture needle 150 relative to the ultrasonic probe 2 immediately before insertion. The puncture needle 150 is initially located at the distal end of the puncture adapter 22. That is, initial needlepoint position information is the position information of the distal end of the puncture adapter 22 relative to the ultrasonic probe 2. The position information storage unit 7 also stores the needlepoint position information and initial needlepoint position information calculated by the position information calculation unit 6.

As shown in FIG. 1, the ultrasonic diagnostic apparatus 100 further includes an insertion area setting unit 8, an expansion image generation unit 9, and an MPR image generation unit 10.

The insertion area setting unit 8 sets an image area in a predetermined range having the planned insertion route of the puncture needle 150 as a central axis in volume data. This image area will be referred to as an insertion area hereinafter. More specifically, the insertion area setting unit 8 sets the planned insertion route of the puncture needle 150 in volume data based on initial needlepoint position information and a puncture target region. For example, a puncture target region is set in accordance with the instruction issued by the operator via an input unit 15 with respect to an MPR image. The insertion area setting unit 8 sets, as an insertion area, an image area having a predetermined size and a predetermined shape with a planned insertion route as a central axis. The insertion area may have a cylindrical shape or polygonal column shape. Assume that an insertion area has a cylindrical shape. The operator can arbitrarily set the radius of an insertion area via the input unit 15.

The expansion image generation unit 9 generates an image expressing the brightness value distribution on a side surface of an insertion area in volume data by the two-dimensional polar coordinates defined by the rotational angle of the insertion area around the central axis and the distance from a reference point on the central axis. This image will be referred to as an expansion image hereinafter.

The MPR image generation unit 10 generates MPR (Multi Planar Reconstruction) image data of a desired slice based on volume data.

The ultrasonic diagnostic apparatus 100 includes a puncture support image generation unit 11. The unit 11 generates an expansion image on which a puncture indicator for supporting the grasping of the position of the puncture needle 150 is superimposed upon positional alignment. An expansion image on which a puncture indicator is superimposed will be referred to as a puncture support image hereinafter.

As shown in FIG. 1, the ultrasonic diagnostic apparatus 100 further includes a display unit 14, the input unit 15, and a system controller 16.

The display unit 14 displays various types of information. For example, the display unit 14 displays MPR images, expansion images, and puncture support images. More specifically, the display unit 14 includes a display data generation unit, a data conversion unit, and a monitor (which are not shown). The display data generation unit generates display data by converting the above MPR image or puncture support image into data in a predetermined display format. The data conversion unit performs conversion processing such as D/A conversion and television format conversion for the above display data. The monitor displays the display data after conversion processing.

The input unit 15 accepts various types of instructions from the operator via an input device. The input device includes, for example, a display panel on an operation panel, a keyboard, a trackball, a mouse, selection buttons, and input buttons.

The system controller 16 comprehensively controls the respective units described above. The system controller 16 includes a CPU and an input information storage unit (which are not shown). The input information storage unit saves the above various types of information input or set by the input unit 15. The CPU comprehensively controls the respective units of the ultrasonic diagnostic apparatus 100 by using the above various types of information. Comprehensively controlling the respective units will execute ultrasonic scanning for a three-dimensional region in the patient. In addition, comprehensively controlling the respective units will execute generation and display of a puncture support image effective for examination or medical treatment using the puncture needle 150 based on the volume data acquired by ultrasonic scanning.

The processing of generating volume data from ultrasonic scanning will be described next.

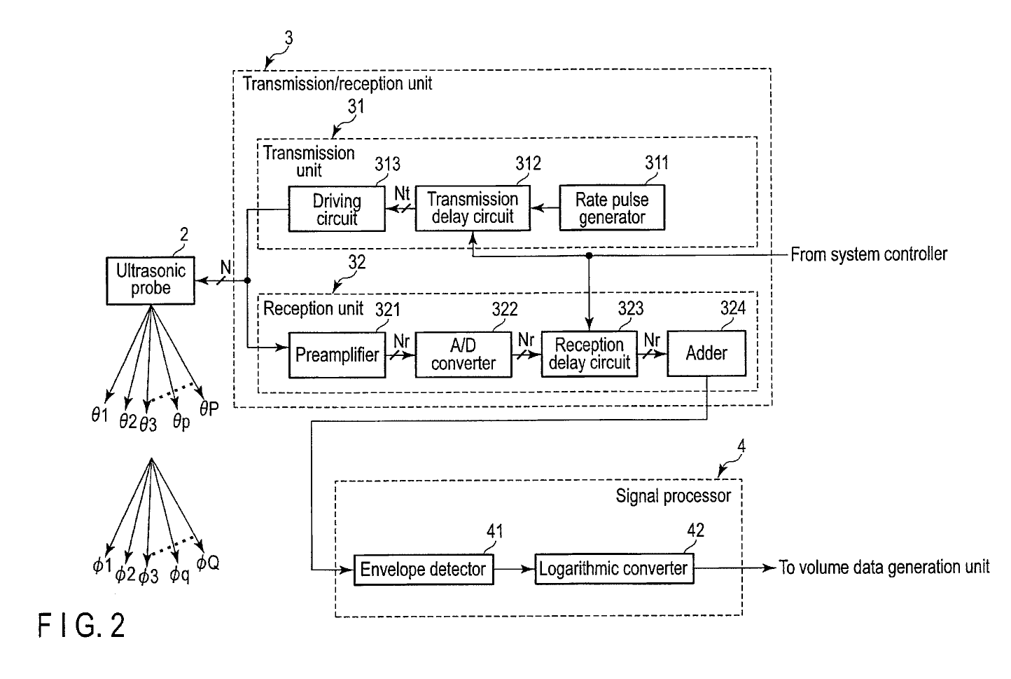

FIG. 2 shows the detailed arrangements of the transmission/reception unit 3 and signal processor 4. The ultrasonic probe 2 has N (N=N1×N2) transducers (not shown) arrayed two-dimensionally at its distal end portion. When performing ultrasonic scanning, the operator brings the distal end portion of the ultrasonic probe 2 into contact with the body surface of a patient. The respective transducers are connected to the transmission/reception unit 3 via an N-channel multicore cable (not shown). These transducers are electroacoustic conversion elements, which convert driving signals (electrical pulses) into transmission ultrasonic waves (ultrasonic pulses) at the time of transmission, and convert reception ultrasonic waves (reflected ultrasonic waves) into electrical reception signals at the time of reception.

Note that the ultrasonic probe 2 includes various types of probes such as probes compatible with sector scanning, linear scanning, and convex scanning. The operator can arbitrarily select a suitable ultrasonic probe 2 in accordance with an examination/medical treatment region. This embodiment will be described on the assumption that the ultrasonic probe 2 is compatible with sector scanning and includes N transducers arrayed two-dimensionally at its distal end portion.

As shown in FIG. 2, the transmission/reception unit 3 includes a transmission unit 31 and a reception unit 32. The transmission unit 31 supplies driving signals for radiating ultrasonic waves in a predetermined direction in the body of a patient to a plurality of transducers included in the ultrasonic probe 2. The reception unit 32 performs phasing addition of the reception signals supplied from the plurality of transducers via a plurality of channels.

The transmission unit 31 includes a rate pulse generator 311, a transmission delay circuit 312, and a driving circuit 313.

The rate pulse generator 311 generates rate pulses for deciding the repetition period of transmission ultrasonic waves radiated into the body by frequency-dividing the reference signal supplied from the system controller 16. The rate pulse generator 311 supplies generated rate pulses to the transmission delay circuit 312. The transmission delay circuit 312 is constituted by, for example, independent delay circuits equal in number to Nt transmission transducers selected from N transducers built in the ultrasonic probe 2. The transmission delay circuit 312 gives convergence delay times and deflection delay times to the above rate pulses supplied from the rate pulse generator 311. Convergence delay times are delay times for the convergence of transmission ultrasonic waves to a predetermined depth. Deflection delay times are delay times for the radiation of ultrasonic transmission waves in a predetermined direction. The driving circuit 313 generates driving pulses to which the above convergence delay times and deflection delay times are given based on the rate pulses supplied from the transmission delay circuit 312. The generated driving pulses are supplied to the Nt transmission transducers built in the ultrasonic probe 2.

The reception unit 32 includes preamplifiers 321, an A/D converter 322, a reception delay circuit 323, and an adder 324.

The preamplifiers 321 are provided in number equal to Nr channels corresponding to Nr reception transducers selected from the N transducers built in the ultrasonic probe 2. The preamplifiers 321 amplify reception signals from the reception transducers. The A/D converter 322 converts reception signals supplied from the preamplifier 321 via the Nr channels into digital signals. The reception delay circuit 323 gives focus delay times and deflection delay times to the respective reception signals output from the A/D converter 322 via the Nr channels. Focus delay times are delay times for the focus of reception ultrasonic waves from a predetermined depth. Deflection delay times are delay times for setting strong reception directivity in a predetermined direction. The adder 324 adds and combine reception signals output from the reception delay circuit 323 via the Nr channels. That is, the reception delay circuit 323 and the adder 324 perform phasing addition of reception signals.

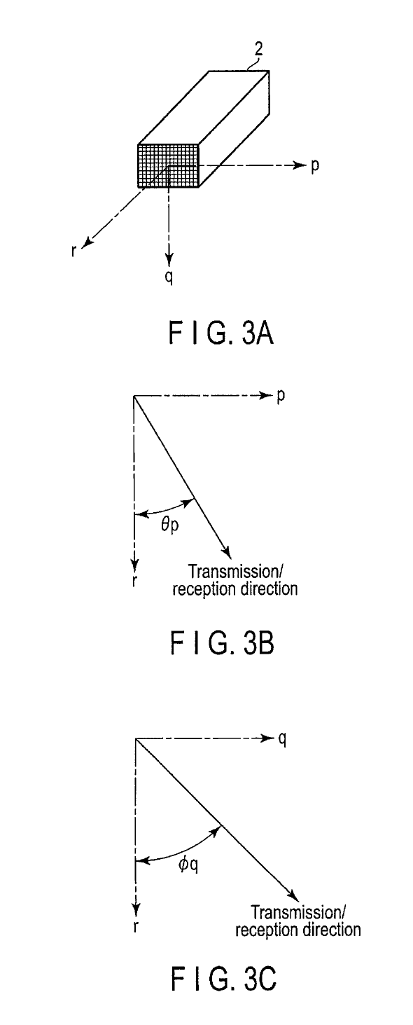

FIG. 3 shows transmission/reception directions Op and θq of ultrasonic waves in an orthogonal coordinate system (pqr), with the r-axis being the central axis of the ultrasonic probe 2. FIG. 3A shows the positional relationship between the ultrasonic probe 2 and the pqr orthogonal coordinate system. Referring to FIG. 3A, for example, N transducers are arrayed two-dimensionally in the p-axis direction and the q-axis direction. The two-dimensional plane defined by the p-axis and the q-axis coincides with the array plane of the N transducers. The r-axis is perpendicular to the array plane of the transducers. The r-axis is defined to pass through the center of the array plane of the transducers. FIG. 3B shows the transmission/reception direction θp of ultrasonic waves projected on the pr plane. FIG. 3C shows the transmission/reception direction φq of ultrasonic waves projected on the qr plane.

As shown in FIG. 2, the reception signal processor 4 includes an envelope detector 41 and a logarithmic converter 42. The envelope detector 41 performs envelope detection of each reception signal output from the adder 324. The logarithmic converter 42 performs logarithmic conversion processing for the reception signals having undergone envelope detection to relatively enhance smaller signal amplitudes. The reception signal having undergone logarithmic conversion processing is called B-mode data. The B-mode data is supplied to the volume data generation unit 5.

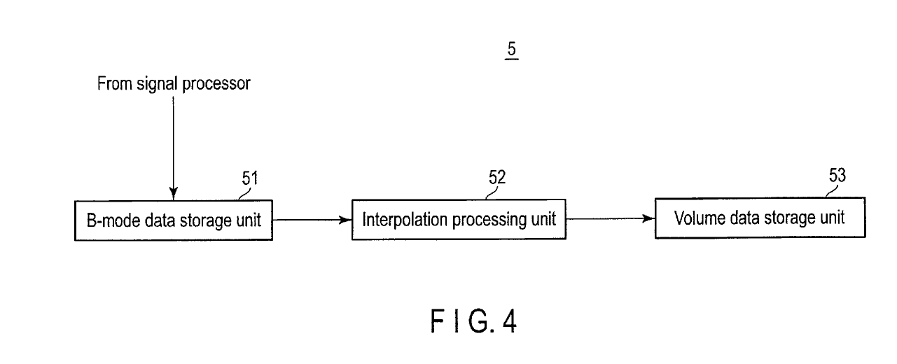

FIG. 4 shows the detailed arrangement of the volume data generation unit 5. The unit 5 includes a B-mode data storage unit 51, an interpolation processing unit 52, and a volume data storage unit 53.

The B-mode data storage unit 51 sequentially stores the B-mode data acquired by ultrasonic scanning in association with the information of the transmission/reception directions θp and θq. The system controller 16 supplies the information of transmission/reception directions.

The interpolation processing unit 52 arrays the B-mode data read out from the B-mode data storage unit 51 in the memory in accordance with the transmission/reception directions θp and θq. The interpolation processing unit 52 generates volume data (B-mode volume data) by performing interpolation processing or the like for the arrayed B-mode data. The volume data storage unit 53 stores the obtained volume data.

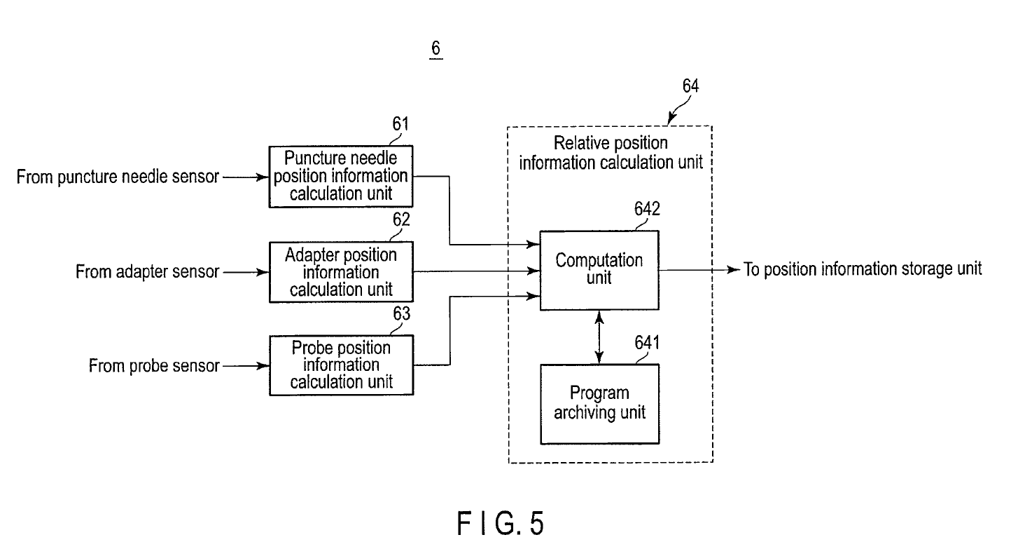

The position information calculation unit 6 will be described in detail next. FIG. 5 shows the detailed arrangement of the position information calculation unit 6. As shown in FIG. 5, the position information calculation unit 6 includes a puncture needle position information calculation unit 61, an adapter position information calculation unit 62, a probe position information calculation unit 63, and a relative position information calculation unit 64. The puncture needle position information calculation unit 61 calculates the position information of the distal end of the puncture needle 150 based on the position signal supplied from the puncture needle sensor 151. The adapter position information calculation unit 62 calculates the position information of the distal end of the puncture adapter 22 (i.e., the position information of the distal end portion of the puncture needle before insertion) based on the position signal supplied from the adapter sensor 23. The probe position information calculation unit 63 calculates the position information (position and direction) of the ultrasonic probe 2 based on the position signals supplied from the plurality of probe sensors 21 provided in or around the ultrasonic probe 2.

Various types of methods of calculating the positions of the puncture needle 150, puncture adapter 22, and ultrasonic probe 2 have already been proposed. A method using an ultrasonic sensor or magnetic sensor as a position sensor is suitably used in consideration of detection accuracy, cost, and size. The probe position information calculation unit 63 using a magnetic sensor is disclosed in, for example, Jpn. Pat. Apple. KOKAI Publication No. 2000-5168. That is, the unit 63 includes a transmitter (magnetism generation unit) and a calculation unit. The transmitter (magnetism generation unit) generates magnetism. The calculation unit calculates the position information (position and direction) of the ultrasonic probe 2 by processing the position signals supplied from a plurality of magnetic sensors (probe sensors 21) which have detected the generated magnetism.

The magnetic sensors used as the probe sensors 21 are generally attached to the surface of the ultrasonic probe 2, and the transmitter of the probe position information calculation unit 63 is placed near the ultrasonic probe 2. The above calculation unit calculates the position and direction of the ultrasonic probe 2 based on the array intervals of the plurality of magnetic sensors and the distances between the respective magnetic sensors and the transmitter which are measured by using magnetism.

As shown in FIG. 5, the relative position information calculation unit 64 includes a program archiving unit 641 and a computation unit 642. The program archiving unit 641 archives a relative position information calculation program. The computation unit 642 performs predetermined computation processing by using the relative position information calculation program.

More specifically, the computation unit 642 calculates the needlepoint position information of the puncture needle 150 inserted into the body of a patient based on the position information of the distal end portion of the puncture needle supplied from the unit 61 and the position information of the ultrasonic probe 2 supplied from the unit 63.

Likewise, the computation unit 642 calculates initial needlepoint position information based on the position information of the puncture adapter 22 supplied from the unit 62 and the position information of the ultrasonic probe 2 supplied from the unit 63.

Needlepoint position information and initial needlepoint position information allow to associate the distal end portion of the puncture needle 150 before insertion or after being inserted into the body of the patient with volume data or the MPR image data based on the volume data.

The position information storage unit 7 in FIG. 1 stores needlepoint position information and initial needlepoint position information. That is, the unit 7 sequentially stores the needlepoint position information repeatedly supplied from the relative position information calculation unit 64 as the distal end of the puncture needle 150 inserted into the body of the patient moves. Likewise, the unit 7 stores the initial needlepoint position information supplied from the relative position information calculation unit 64 as the position/direction of the puncture adapter 22 is set or updated.

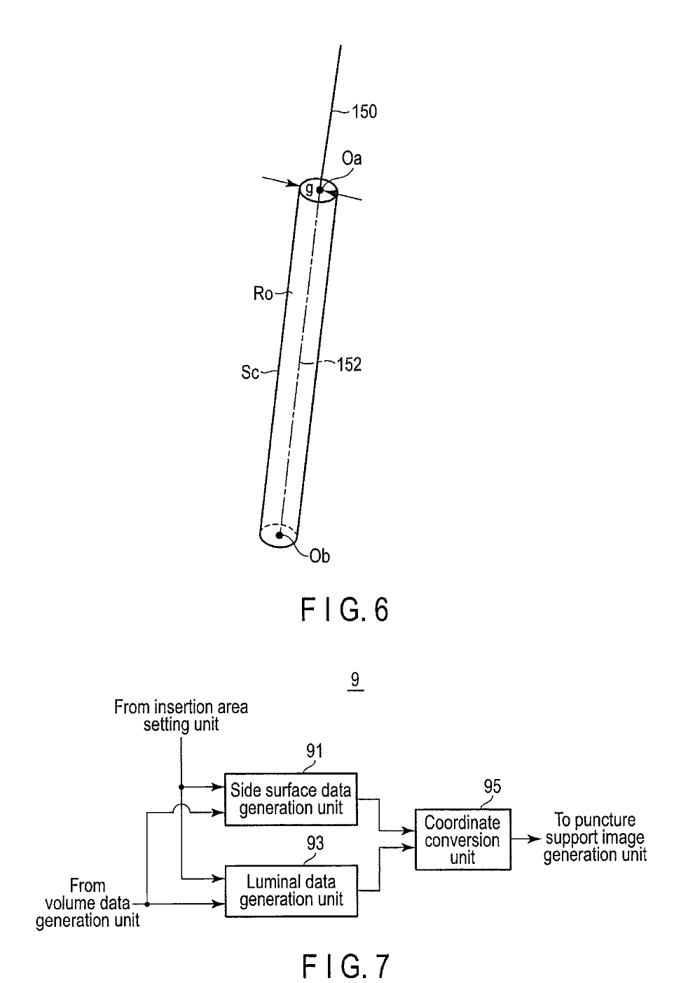

The insertion area setting unit 8 will be described in detail next with reference to FIG. 6. FIG. 6 is a view schematically showing an insertion area Ro. As shown in FIG. 6, the insertion area Ro has a central axis 152 indicated by the one-dotted dashed line. The central axis 152 is set on a line segment connecting an initial position Oa of the distal end of the puncture needle 150 to a reference point Ob in a puncture target region. The initial position Oa is uniquely decided by the position information (position and inclined angle) of the puncture adapter 22. The reference point Ob can be set at an arbitrary point such as the central point, barycentric point, or end point of a puncture target region. The central axis 152 coincides with the planned insertion route. The insertion area Ro is a cylindrical image area having a preset value g as a radius. A side surface Sc is defined on the insertion area Ro.

In insertion area setting processing, the insertion area setting unit 8 reads out initial needlepoint position information from the position information storage unit 7. The insertion area setting unit 8 waits until the operator inputs a puncture target region via the input unit 15. The insertion area setting unit 8 sets the insertion area Ro in volume data based on initial needlepoint position information corresponding to the initial position Oa and the position information of the reference point Ob of the puncture target region. More specifically, first of all, the insertion area setting unit 8 sets a planned insertion route in the volume data based on the initial needlepoint position information and the position information of the reference point Ob. Note that the positions of the ultrasonic probe 2 and puncture adapter 22 are adjusted in advance such that the insertion start position based on the initial needlepoint position information and the reference point Ob of the patient pass through the planned insertion route. If, for example, the angle of a planned insertion route is uniquely decided by the position and inclined angle of the puncture adapter 22, the positions of the ultrasonic probe 2 and puncture adapter 22 are adjusted to make the planned insertion route intersect with the reference point Ob. It is preferable to perform this position adjustment under the observation of an MPR image indicating a planned insertion route and a puncture target region. This determines a planned insertion route. The insertion area setting unit 8 then sets a linear line segment connecting the puncture start position (initial puncture needle position) and the reference point Ob of the puncture target region, i.e., a planned insertion route, at the central axis 152 of the insertion area Ro in accordance with an instruction given by the operator via the input unit 15.

The processing performed by the expansion image generation unit 9 will be described next. FIG. 7 shows the detailed arrangement of the unit 9. As shown in FIG. 7, the unit 9 includes a side surface data generation unit 91, a luminal data generation unit 93, and a coordinate conversion unit 95.

The side surface data generation unit 91 extracts a plurality of voxels existing on a side surface of an insertion area from volume data from the volume data generation unit 5. A set of a plurality of voxels existing on a side surface of an insertion area will be referred to as side surface data.

The luminal data generation unit 93 extracts a plurality of voxels concerning a predetermined anatomical region from volume data. As an extraction target anatomical region, for example, a luminal organ such as a blood vessel or digestive tract is suitable. For example, the unit 93 extracts voxels corresponding an extraction target luminal organ by comparing the voxel values of volume data existing in an insertion area with a predetermined threshold. As a predetermined threshold, a typical brightness value of voxels corresponding to an extraction target luminal organ is used. A set of a plurality of voxels existing in a luminal organ will be referred to as luminal data.

The coordinate conversion unit 95 generates an expansion image based on side surface data. More specifically, the unit 95 generates expansion image by performing coordinate conversion of side surface data in accordance with a predetermined conversion rule. The conversion rule is a coordinate conversion expression for converting an orthogonal three-dimensional coordinate system into a two-dimensional polar coordinate system defining an expansion image. The unit 95 may perform coordinate conversion of brightness data in accordance with the same conversion rule as that used for side surface data and superimpose a luminal region corresponding to the luminal data after coordinate conversion on an expansion image.

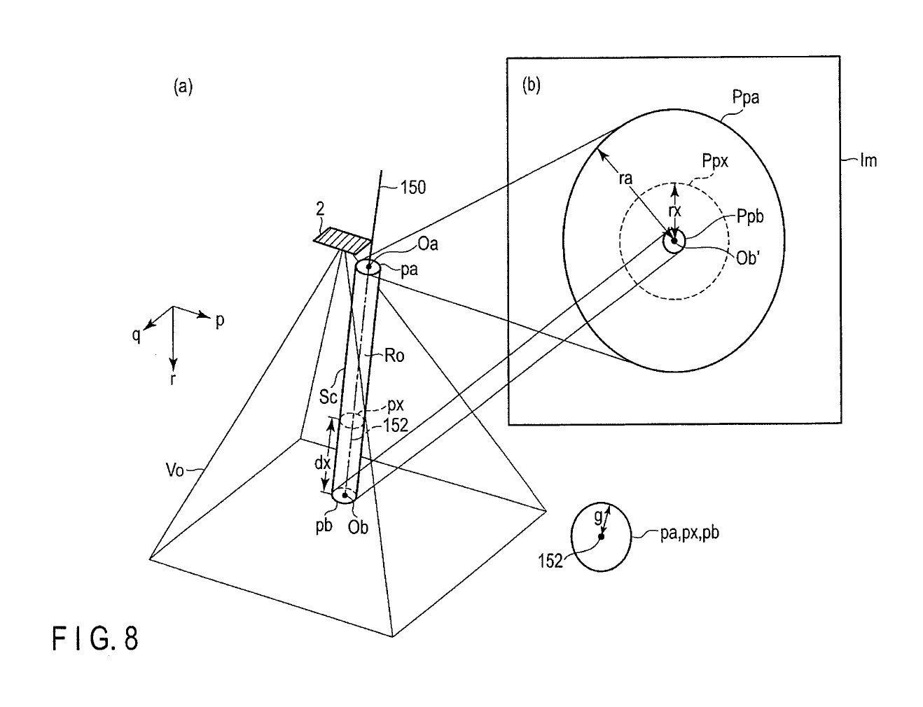

FIG. 8 is a view for explaining a method of generating an expansion image Im. In FIG. 8, (a) indicates an insertion area Ro set in volume data Vo. In FIG. 8, (b) indicates the expansion image Im concerning a side surface Sc of the insertion area Ro. As shown in FIG. 8, the expansion image Im has a two-dimensional polar coordinate system defined by a rotational angle around a central axis 152 and the distance from a reference point Ob on the central axis 152, with the reference point Ob in a puncture target region being an origin. An intersecting line px between a plane perpendicular to the central axis 152 and the side surface Sc, which is located at a distance dx from the reference point Ob, corresponds to a concentric circle Ppx centered on an origin Ob′ of the expansion image Im and located at a distance rx. The origin Ob′ corresponds to the reference point Ob. The expansion image generation unit 9 assigns the brightness values of a plurality of voxels on the intersecting line px to a plurality of pixels on the concentric circle Ppx in the expansion image Im. Repeating this assigning processing while changing the distances dx and rx will generate the expansion image Im. For the sake of simplicity, (b) in FIG. 8 indicates no variation in brightness value in the expansion image Im. In practice, however, variations in brightness values corresponding to the brightness value distribution on a side surface of the insertion area Ro are displayed in the expansion image.

The display unit 14 displays the generated expansion image. As described above, the expansion image indicates the morphological information of the puncture needle 150 in the entire circumferential direction around the planned insertion route (the central axis of the insertion area). The operator can therefore observe anatomical information around the planned insertion route in one window. In contrast, conventional ultrasonic diagnostic apparatuses have been configured to support the insertion of a puncture needle by displaying an MPR image depicting a planned insertion route. If the puncture needle deviates from the planned insertion route, no MPR image is depicted in the puncture needle region. Depicting no puncture needle region in the MPR image makes the operator feel anxiety. In practice, it is not always necessary for the puncture needle 150 to reach a puncture target region without deviating from a planned insertion route at all. The puncture needle 150 may deviate from the planned insertion route as long as it reaches the puncture target region. As described above, the expansion image includes a puncture target region although including no planned insertion route. For this reason, since an MPR image does not depict the unnecessary puncture needle unlike a conventional MPR image, the operator can dedicate himself/herself to the insertion of the puncture needle 150 without feeling any stress originating from temporal deviation of an actual insertion route from the planned insertion route.

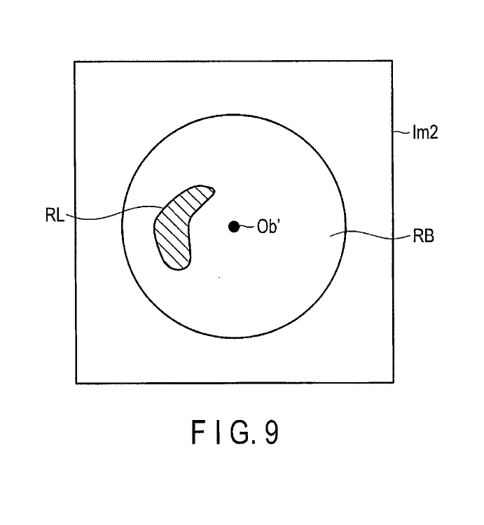

As described above, a luminal region may be superimposed on an expansion image. FIG. 9 shows an example of an expansion image Im2′ on which a luminal region RL is superimposed. As shown in FIG. 9, the luminal region RL is superimposed on an expansion image Im2. A remaining image region RB included in the expansion image Im2 originates from side surface data. The display unit 14 displays the luminal region RL and the remaining image region RB while visually discriminating them from each other. For example, the display unit 14 displays the luminal region RL and the remaining image region RB in different colors. This allows the operator to clearly grasp the existence range of the luminal region RL in the expansion image Im2.

Note that the operator can arbitrarily make settings via the input unit 15 to determine whether to superimpose a luminal region on an expansion image.

The processing performed by the puncture support image generation unit 11 will be described next. The unit 11 generates puncture indicators and superimposes the generated puncture indicators on an expansion image upon positional alignment, thereby generating a puncture support image.

Puncture indicators include, for example, a distance mark. A distance mark is a mark which indicates distances from a reference point on a planned insertion route on an expansion image at predetermined intervals. A reference point is set at the origin of an expansion image, i.e., a reference point in a puncture target region.

The processing of generating a puncture support image including a distance mark will be described next. The puncture support image generation unit 11 generates a distance mark in accordance with predetermined mark intervals.

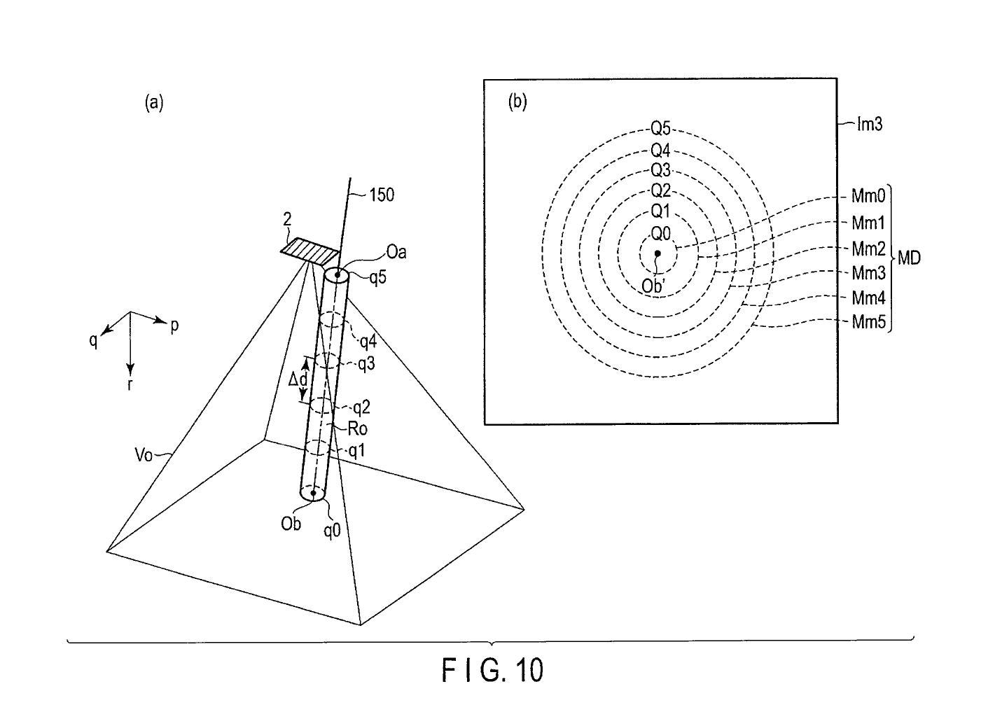

FIG. 10 explains a distance mark. In FIG. 10, (a) schematically shows the volume data Vo, the puncture needle 150, and the insertion area Ro in a real space. In FIG. 10, (b) shows a puncture support image Im3 including a distance mark MD. For example, the operator inputs the value of a mark interval Δd via the input unit 15. The puncture support image generation unit 11 generates the distance mark MD in accordance with the input mark interval Δd. The distance mark MD is constituted by a plurality of scale marks Mm for indicating distances from the reference point Ob in the puncture target region at the mark intervals Δd. For example, let q0 be a position at a distance 0 from the reference point Ob, q1 be a position at a distance Δd, q2 be a position at a distance 2Δd, q3 be a position at a distance 3Δd, q4 be a position at a distance 4Δd, and q5 be a position at a distance 5Δd. In this case, the puncture support image generation unit 11 generates a scale mark Mm0 corresponding to the distance q0, a scale mark Mm1 corresponding to the distance q1, a scale mark Mm2 corresponding to the distance q2, a scale mark Mm3 corresponding to the distance q3, a scale mark Mm4 corresponding to the distance q4, and a scale mark Mm5 corresponding to the distance q5. Each scale mark Mm is formed from a circular line (the dotted line in FIG. 10) centered on the origin Ob′. The radius of each scale mark Mmn (n is an arbitrary integer) corresponds to the distance from the origin to qn. The line type of each scale mark Mm is not limited to a dotted line, and any one of all line types such as a solid line and a one-dotted dashed line may be arbitrarily selected. The puncture support image generation unit 11 combines these scale marks with an expansion image at corresponding positions. This generates the puncture support image Im3 including the distance mark. The display unit 14 displays the generated puncture support image Im3.

In this case, the display unit 14 may explicitly display the position of the distal end portion of the puncture needle 150 on an expansion image. For example, the display unit 14 may change the display form of the distance mark in accordance with the position of the distal end portion of the puncture needle 150.

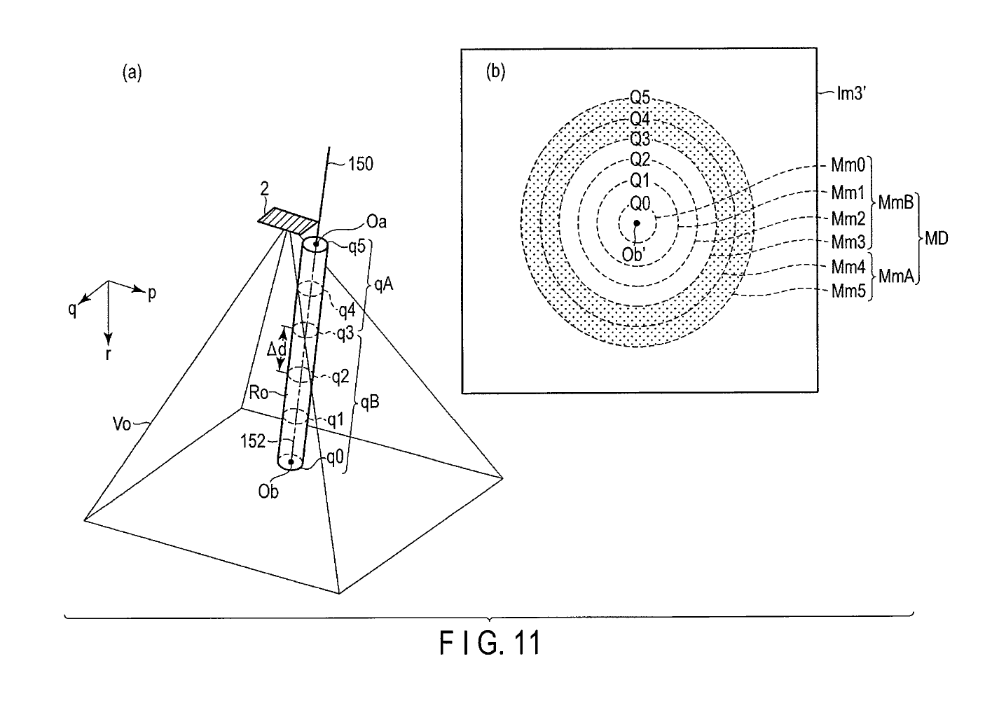

FIG. 11 shows an example of a puncture support image Im3′ including the distance mark MD corresponding to the position of the distal end of the puncture needle 150. In FIG. 11, (a) schematically shows the puncture needle 150 and the insertion area Ro in a real space. In FIG. 11, (b) shows the puncture support image including the distance mark MD. As indicated by (a) in FIG. 11, assume that the distal end of the puncture needle 150 is located in the zone between the distance q3 and the distance q4 along the planned insertion route (central axis 152).

The puncture support image generation unit 11 specifies the zone in which the distal end of the puncture needle 150 is located based on the mark interval Δd and the needlepoint position information. Zones are demarcated by sectioning the insertion area Ro at the mark intervals Δd along the central axis 152. For example, in (a) in FIG. 11, the insertion area is sectioned into a zone [q0−q1], a zone [q1−q2], a zone [q2−q3], a zone [q3−q4], and a zone [q4−q5]. The unit 11 estimates a zone qA through which the distal end of the puncture needle 150 has already passed and a zone qB through which the distal end has not yet passed based on the zone in which the distal end of the puncture needle 150 is located. More specifically, the unit 11 estimates the zone including the zone in which the distal end of the puncture needle 150 is located and the zone closer to the initial position of the puncture needle 150 than the above zone as the zone qA through which the distal end has already passed. The zone closer to the initial position of the puncture needle 150 than the above zone is estimated based on needlepoint position information and initial needlepoint position information. Alternatively, if the history of the positions of the distal end of the puncture needle 150 in the examination is saved, a zone 1A through which the distal end has already passed may be specified by using the history. The unit 11 assigns different visual effects to a distance mark MmA corresponding to the zone qA through which the distal end has already passed and a distance mark MmB corresponding to the zone qB through which the distal end has not yet passed. This allows the display unit 14 to display the distance mark MmA corresponding to the zone qA through which the distal end has already passed and the distance mark MbB corresponding to the zone qB through which the distal end has not yet passed so as to visually discriminate them from each other. For example, different color values are assigned to the distance mark MbB and the distance mark MmB. This allows the display unit 14 to display the distance mark MmA and the distance mark MmB in different colors. Note that the display unit 14 may display the distance mark MmA and the distance mark MmB in different graphic patterns. This allows the operator to roughly grasp the current position of the distal end of the puncture needle 150 on an expansion image (or a puncture support image).

Note that the puncture support image generation unit 11 may assign different visual effects to a distance mark corresponding to the zone in which the distal end of the puncture needle 150 is located and a distance mark corresponding to the remaining zone. This allows the display unit 14 to display the distance mark corresponding to the zone in which the distal end of the puncture needle 150 is located and the distance mark corresponding to the remaining zone so as to visually discriminate from each other. This allows the operator to roughly grasp the current position of the distal end of the puncture needle on an expansion image (or a puncture support image).

For various reasons, the puncture needle 150 sometimes deviates from an insertion area. In this case, since it is highly probable that the puncture needle 150 does not reach the puncture target region, it is preferable to notify the operator to that effect. The puncture support image generation unit 11 can superimpose an indicator indicating the corresponding information on an expansion image. That is, the unit 11 generates a mark indicating the intersecting position between the distal end of the puncture needle 150 inserted into the body of a patient and the side surface of the insertion area. A mark indicating such an intersecting position will be referred to as an intersecting position mark hereinafter.

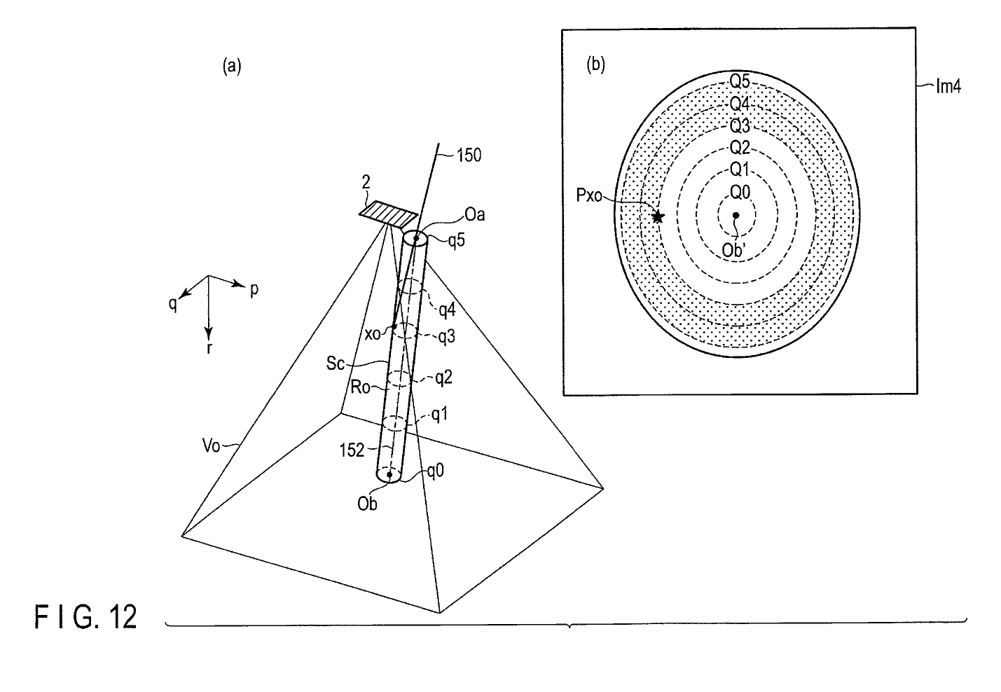

FIG. 12 shows an example of a puncture support image Im4 including an intersecting position mark Pxo. In FIG. 12, (a) schematically shows the volume data Vo, the puncture needle 150, and the insertion area Ro in a real space. In FIG. 12, (b) shows the puncture support image Im4 including the intersecting position mark Pxo. As indicated by (a) in FIG. 12, assume that the distal end of the puncture needle 150 deviates from the planned insertion route (central axis 152) in the zone [q3−q4] and intersects with the side surface Sc of the insertion area Ro.

First of all, the puncture support image generation unit 11 calculates the coordinates of an intersecting position Xo between the side surface Sc of the volume data Vo and the distal end of the puncture needle 150 based on needlepoint position information and the position information of the side surface Sc. The unit 11 calculates the three-dimensional coordinates of the intersecting position Xo defined by the pqr orthogonal coordinate system. The unit 11 calculates polar coordinates Pxo of the puncture support image Im4 which correspond to the calculated three-dimensional coordinates. For example, the puncture support image generation unit calculates the polar coordinates Pxo by applying the above conversion rule to the three-dimensional coordinates. The unit 11 adds the intersecting position mark Pxo to the pixel of the calculated polar coordinates Pxo. This generates the puncture support image Im4 including the intersecting position mark Pxo. The display unit 14 displays the puncture support image Im4. The display unit 14 preferably enhances the intersecting position mark Pxo in the puncture support image Im4 to allow the operator to easily grasp the position of the distal end of the puncture needle 150.

By displaying an intersecting position mark in this manner, the display unit 14 can notify the operator that the puncture needle 150 has intersected with the side surface of the insertion area Ro. This allows the operator to recognize that the insertion route of the puncture needle 150 has greatly deviated from the planned insertion route.

### (Puncture Support Data Generation/Display Procedures)

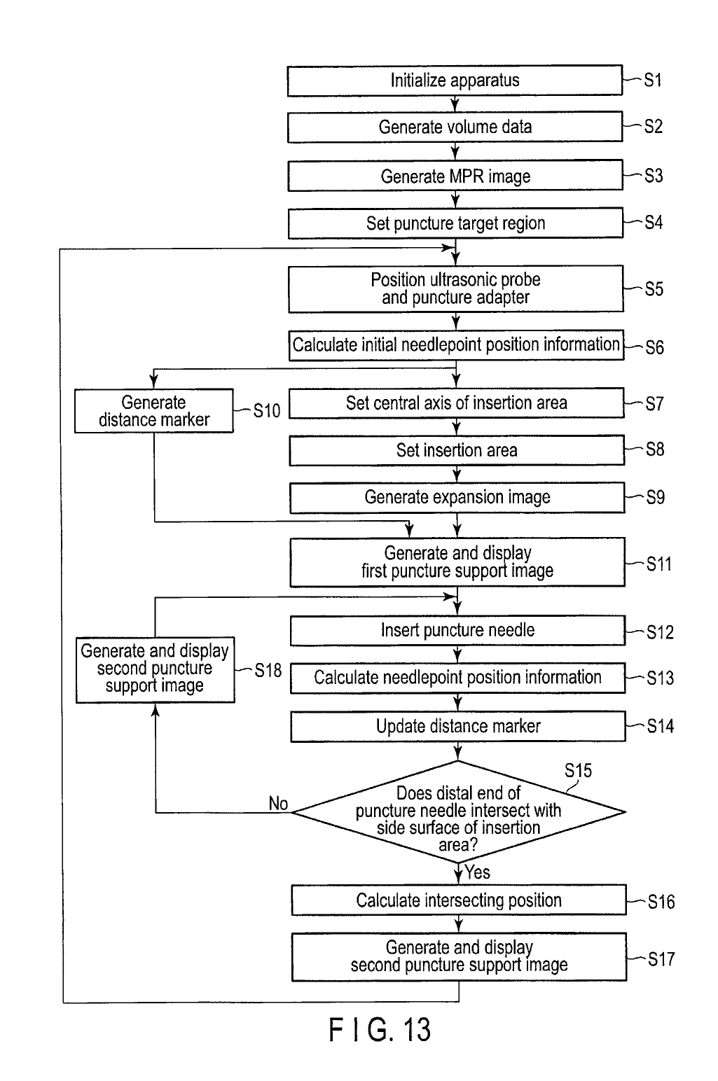

Puncture support data generation/display procedures in this embodiment will be described next with reference to FIG. 13. Before acquiring volume data corresponding to a patient, the operator inputs patient information via the input unit 15. Upon inputting the patient information, the operator sets volume data generation conditions, MPR image data generation conditions, CPR image data generation conditions, luminal data generation conditions, generation conditions, puncture support data generation conditions, a puncture area diameter, a mark interval, an expansion radius, and the like. The input information storage unit of the system controller 16 stores the above input information and setting information input via the input unit 15 (step S1).

Upon completing the above initialization for the ultrasonic diagnostic apparatus 100, the operator inputs, via the input unit 15, a start instruction signal for the generation of a support image while the ultrasonic probe 2 is placed on the body surface of a patient. The input start instruction signal is supplied to the system controller 16. Upon receiving the instruction signal, the system controller 16 starts to acquire volume data concerning a three-dimensional region in the body of the patient including a puncture target region.

When acquiring volume data, the rate pulse generator 311 generates rate pulses in accordance with a control signal from the system controller 16. The generated rate pulses are supplied to the transmission delay circuit 312. The transmission delay circuit 312 gives the rate pulses delay times to converge ultrasonic waves to a predetermined depth so as to obtain a small beam width in transmission and delay times to transmit ultrasonic waves in the first transmission/reception directions θ1 and φ1. The rate pulses to which the delay times have been given are supplied to the N-channel driving circuit 313. The driving circuit 313 then generates driving signals having predetermined delay times and shapes based on the rate pulses supplied from the transmission delay circuit 312. The generated driving signals are supplied to the N transducers in the ultrasonic probe 2. The transducers which have received the driving signals radiate transmission ultrasonic waves into the body of the patient.

The radiated transmission ultrasonic waves are partly reflected by organ interfaces or tissues having different acoustic impedances and received by the transducers. The transducers convert the reflected waves into electrical reception signals. The preamplifiers 321 of the reception unit 32 gain-correct the reception signals. The A/D converter 322 converts the signals into digital signals. The N-channel reception delay circuit 323 gives focus delay times and directivity delay times to the digital signal. By the focus delay times are given to the digital signals, the reception ultrasonic waves from the predetermined depth are signally focused. By the directivity delay times are given to the digital signals, the reception ultrasonic waves from the directions θ1 and φ1 are signally set for strong reception directivity. The adder 324 performs phasing addition of the reception signals to which these delay times have been given.

The reception signal after phasing addition is supplied to the envelope detector 41. The envelope detector 41 performs envelope detection of this reception signal. The reception signal having undergone the envelope detection is supplied to the logarithmic converter 42. The logarithmic converter 42 performs logarithmic conversion of the supplied reception signal to generate B-mode data. The B-mode data storage unit 51 of the volume data generation unit 5 stores the obtained B-mode data in association with the transmission/reception direction (θ1, φ1) information.

Upon completion of the generation and saving of B-mode data in the transmission/reception directions θ1 and φ1, the apparatus performs ultrasonic transmission/reception in transmission/reception directions θ1 and φ2 to φQ set by updating the transmission/reception directions of ultrasonic waves for each Δφ according to φq=φ1+(q−1)Δφ (q=2 to Q). The apparatus further repeats ultrasonic transmission/reception in transmission/reception directions φ1 to φQ described above with respect to transmission/reception directions θ2 to θP set by updating the transmission/reception direction for each Δθ in the θ direction according to θp=θ1+(p−1)Δθ (p=2 to P), thereby performing three-dimensional scanning. The B-mode data storage unit 51 also saves the B-mode data obtained by these ultrasonic transmission/reception operations in association with the above pieces of transmission/reception direction information.

The interpolation processing unit 52 of the volume data generation unit 5 arrays the B-mode data read out from the B-mode data storage unit 51 in accordance with the transmission/reception directions θp and φq to generate three-dimensional B-mode data. The interpolation processing unit 52 generates volume data (B-mode volume data) by performing interpolation processing for the generated three-dimensional B-mode data. The volume data storage unit 53 stores the generated volume data (step S2).

The MPR image generation unit 10 then sets an MPR slice with respect to a lesion (puncture target region) in the volume data read out from the volume data storage unit 53. The MPR image generation unit 10 extracts voxels in the set MPR slice from the volume data to generate an MPR image (step S3). The monitor of the display unit 14 displays the generated MPR image.

The operator observes the MPR image displayed on the display unit 14, and performs operation to set a puncture target region in a lesion via the input device of the input unit 15. In accordance with this operation, the insertion area setting unit 8 sets a reference point in a puncture target region in the lesion in the volume data (step S4).

The operator further positions the ultrasonic probe 2 and the puncture adapter 22 such that the planned insertion route displayed on the display unit 14 intersects with the above puncture target region (step S5).

At this time, the probe position information calculation unit 63 calculates the position information (position and direction) of the ultrasonic probe 2 placed on the body surface of the patient based on the position signal supplied from the probe sensors 21. The adapter position information calculation unit 62 calculates the position information of the distal end of the puncture adapter 22 placed near the body surface of the patient based on the position signal supplied from the adapter sensor 23.

The relative position information calculation unit 64 calculates initial needlepoint position information based on the position information of the distal end of the puncture adapter 22 supplied from the adapter position information calculation unit 62 and the position information of the ultrasonic probe 2 supplied from the probe position information calculation unit 63 (step S6). The position information storage unit 7 stores the initial needlepoint position information.

The insertion area setting unit 8 sets the central axis of the insertion area in the volume data based on the initial needlepoint position information read out from the position information storage unit 7 and the position information of a reference point in the puncture target region (step S7). The insertion area setting unit 8 sets an insertion area based on the position information of the set central axis and the radius information input via the input unit 15 (step S8).

In response to the setting of the insertion area, the side surface data generation unit 91 generates side surface data by extracting voxels in volume data existing on the side surface of the insertion area.

The luminal data generation unit 93 extracts voxels corresponding to a luminal organ by comparing the voxel values of volume data existing in the insertion area with a predetermined threshold, and generates luminal data based on these voxels.

The coordinate conversion unit 95 generates an expansion image by converting the coordinates of the side surface data and luminal data according to a predetermined conversion rule (step S9).

The puncture support image generation unit 11 generates a distance mark indicating the distance from the puncture target region to the initial needlepoint position information based on initial needlepoint position information, the position information of the puncture target region, and mark intervals (step S10).

The puncture support image generation unit 11 generates a first puncture support image by superimposing the distance mark on the expansion image. The display unit 14 displays the generated first puncture support image (step S11).

The operator inserts the distal end portion of the puncture needle 150, which is slidably attached to the puncture adapter 22, into the body of the patient under the observation of the first puncture support image displayed on the display unit 14 (step S12).

The puncture needle position information calculation unit 61 calculates the position information of the distal end of the puncture needle 150 based on the position signal supplied from the puncture needle sensor 151. The relative position information calculation unit 64 calculates the needlepoint position information based on the position information of the ultrasonic probe 2 which is supplied from the probe position information calculation unit 63 and the position information of the distal end of the puncture needle 150 which is supplied from the puncture needle position information calculation unit 61 (step S13).

The puncture support image generation unit 11 updates the distance mark by adding the needlepoint position information supplied from the position information calculation unit 6 to the distance mark generated in step S10 described above (step S14). The puncture support image generation unit 11 determines, based on the above needlepoint position information and the position information of the side surface of the insertion area whether the side surface of the insertion area intersects with the puncture needle 150 (step S15). Upon determining that they intersect with each other, the puncture support image generation unit 11 calculates the intersecting position (step S16).

The puncture support image generation unit 11 generates a second puncture support image by superimposing the updated distance mark and an intersecting position mark on the expansion image supplied from the expansion image generation unit 9. The display unit 14 displays the generated second puncture support image (step S17).

The operator observes the second puncture support image displayed on the display unit 14. Upon observation, the operator may recognize that the inserting direction of the puncture needle 150 is inappropriate. In this case, the operator repeatedly positions the ultrasonic probe 2 and the puncture adapter 22 until the puncture needle 15 stops intersecting with the side surface of the insertion area (step S5). When the operator performs positioning again, the apparatus repeats the processing in step S6 and the subsequent steps under the control of the system controller 16.

Upon determining in step S15 that the puncture needle 150 does not intersect with the side surface of the insertion area, the puncture support image generation unit 11 generates a second puncture support image by superimposing the updated distance mark on the expansion image. The display unit 14 displays the second puncture support image (step S18).

Upon determining that the inserting direction of the puncture needle 150 is appropriate upon observing the second puncture support image displayed on the display unit 14, the operator keeps inserting the distal end portion of the puncture needle toward the puncture target region (step S12). The apparatus repeats the processing in step S13 and the subsequent steps under the control of the system controller 16 as the operator inserts the puncture needle 150.

This is the end of the description of an example of the operation of the ultrasonic diagnostic apparatus 100 according to this embodiment.

According to this embodiment, when inserting the puncture needle 150 into a puncture target region in the body of a patient, the operator can accurately grasp forward information and surrounding information of the distal end of the puncture needle 150 before or during insertion. This makes it possible to efficiently perform safe puncturing operation with respect to the patient.

The ultrasonic diagnostic apparatus 100, in particular, displays an expansion image generated by expanding a brightness value distribution on the side surface of the insertion area, with the planned insertion route of the puncture needle 150 being a central axis, into polar coordinates. Observing the expansion image allows the operator to accurately grasp the state of the region into which the operator can insert the puncture needle 150. The ultrasonic diagnostic apparatus 100 also displays the expansion image upon superimposing, on it, a luminal region such as a blood vessel or digestive organ which is separately generated. Grasping this expansion image allows the operator to estimate an insertion difficulty level until insertion to the puncture target region in advance.

In addition, the ultrasonic diagnostic apparatus 100 displays the above expansion image upon superimposing the distance mark on it. Observing this expansion image allows the operator to accurately measure the distance from the distal end of the puncture needle 150 before or during insertion to the puncture target region. The ultrasonic diagnostic apparatus 100 can also display a distance mark corresponding to a zone through which the distal end of the puncture needle 150 has passed and a distance mark corresponding to the remaining zone so as to visually discriminate the regions from each other. Observing this expansion image allows the operator to accurately grasp the position (insertion depth) of the distal end of the puncture needle 150 in the insertion area.

The ultrasonic diagnostic apparatus 100 detects whether the puncture needle 150 intersects with the side surface of the insertion area. Upon detecting that they intersect with each other, the ultrasonic diagnostic apparatus 100 superimposes a mark at the intersecting position in the expansion image. Observing this expansion image allows the operator to easily determine whether it is necessary to insert the puncture needle again.

Note that this embodiment is not limited to the embodiment described above and can be modified and executed.

In the above embodiment, volume data is generated based on B-mode data. However, this embodiment is not limited to this. The ultrasonic diagnostic apparatus 100 may generate the above volume data based on other ultrasonic data such as color Doppler data.

The above embodiment has exemplified the case in which a puncture target region is set by using an MPR image. However, this embodiment is not limited to this. The ultrasonic diagnostic apparatus 100 may set a puncture target region by using a three-dimensional image such as volume rendering image generated based on the volume data.

The above embodiment has exemplified the case in which the position information of the distal end portion of the puncture needle is detected by using an ultrasonic sensor or magnetic sensor. However, this embodiment is not limited to this. The ultrasonic diagnostic apparatus 100 may detect the position information of the distal end of the puncture needle 150 by extracting the distal end of the puncture needle 150 displayed on an MPR image or three-dimensional image by image processing or the like.

The above embodiment has exemplified the case in which initial needlepoint position information is calculated based on the position information of the distal end of the puncture adapter 22. However, this embodiment is not limited to this. For example, initial needlepoint position information may be calculated based on the position information of the distal end of the puncture needle 150 before insertion.

The above embodiment has exemplified the case in which the planned insertion route of the puncture needle 150 is decided based on the position information of the puncture adapter 22, and the positions and directions of the ultrasonic probe 2 and puncture adapter 22 are adjusted to make the planned insertion route coincide with the puncture target region. However, this embodiment is not limited to this. For example, a plurality of position sensors may be placed on the distal end portion and the like of the puncture needle 150 to decide a planned insertion route based on the position signals supplied from the plurality of position sensors.

Application examples of this embodiment will be described below.

### Application Example 1

The expansion image generation unit 9 in the above embodiment generates an expansion image concerning a side surface of an insertion area having, as a central axis, a line segment extending from a puncture target region to an initial needlepoint position along a planned insertion route. However, this embodiment is not limited to this. The expansion image generation unit 9 may generate an expansion image concerning a side surface of an insertion area having, as a central axis, a line segment extending from a puncture needlepoint position to a specific position along a planned insertion route. In other words, the unit 9 may limit the radial direction of an expansion image to the range from the puncture needlepoint position to a specific position. The puncture support image generation unit 11 can generate a puncture support image based on the expansion image with such radial direction range being limited.

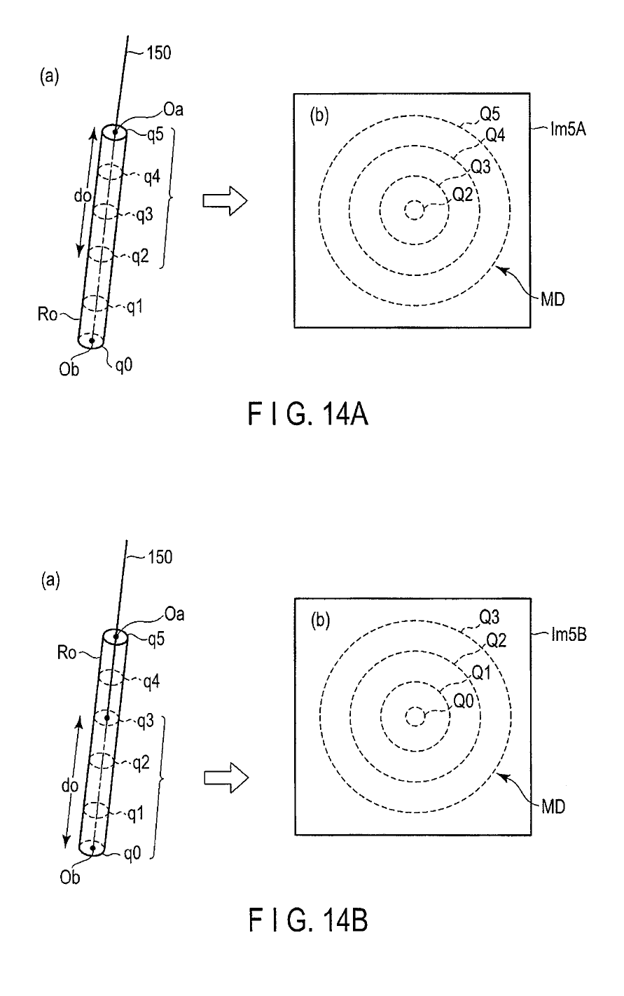

FIGS. 14A and 14B show a puncture support image according to Application Example 1. FIG. 14A is a view for explaining a puncture support image Im5A concerning zones [q2 to q5]. In FIG. 14A, (a) shows the positional relationship between the puncture needle 150 and the insertion area Ro while the distal end of the puncture needle 150 is located at the initial position Oa. In FIG. 14A, (b) shows the puncture support image Im5A concerning the zones [q2 to q5]. The puncture support image (expansion image) Im5A is an image expressing the brightness value distribution on the side surface of an insertion area by using the above two-dimensional polar coordinates, with a line segment extending from the puncture needlepoint position to a predetermined distance do being a central axis. As indicated by (b) in FIG. 14A, the distance mark MD concerning zones from the distal end of the puncture needle 150 to the predetermined distance do is superimposed on the puncture support image Im5A. FIG. 14B is a view for explaining a puncture support image Im5B concerning zones [q0 to q3]. In FIG. 14B, (a) shows the positional relationship between the puncture needle 150 and the insertion area Ro while the distal end of the puncture needle 150 is located at the distance do from the reference point Ob. In FIG. 14B, (b) shows the puncture support image Im5B concerning the zones [q0 to q3]. The puncture support image (expansion image) Im5B is an image expressing the brightness value distribution on the side surface of the insertion area Ro, with a line segment extending from the distal end of the puncture needle 150 to the reference point Ob being a central axis, by the above two-dimensional polar coordinates. As indicated by (b) in FIG. 14B, the distance mark MD concerning the zones from the distal end of the puncture needle 150 to the predetermined distance do is superimposed on the puncture support image (expansion image) Im5B. The operator can arbitrarily set the predetermined distance do via the input unit 15. Assume that the mark intervals in (a) in FIG. 14A and (a) in FIG. 14B are the same as the mark intervals dx in FIG. 10.

The puncture support image generation unit 11 may set a larger display magnification for an expansion image having a narrow display range in the radial direction than that for an expansion image having a wide display range in the radial direction. This allows the operator to observe a region near the distal end portion of the puncture needle with higher accuracy.

The expansion image generation unit 9 updates the expansion image according to Application Example 1 every time the operator moves the distal end of the puncture needle 150. As described above, according to Application Example 1, it is possible to display a brightness value distribution in a predetermined distance range from the distal end of the puncture needle 150 by using an expansion image in real time. This allows the operator to observe a realistic expansion image with his/her gaze fixed on the distal end of the puncture needle 150.

An expansion image according to Application Example 1 can be variously modified. For example, the apparatus may superimpose the puncture target region set by the user on the expansion image according to Application Example 1.

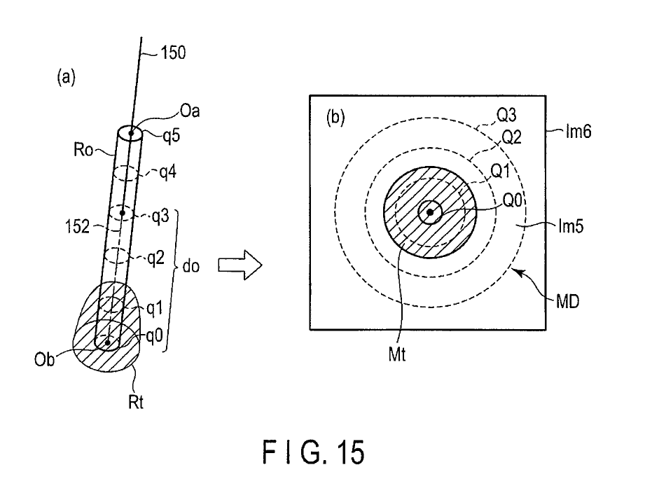

FIG. 15 shows a puncture support image Im6 including an expansion image Im5 on which a puncture target region Rt is superimposed. In FIG. 15, (a) shows the insertion area Ro and the puncture target region Rt in the volume data. The central axis 152 of the insertion area Ro is set at a line segment connecting the reference point Ob of the puncture target region Rt to the initial position Oa of the puncture needle 150. Assume that the distal end of the puncture needle 150 in FIG. 15 has reached the position q3 at the predetermined distance do from the reference point Ob. The distance mark MD concerning the zones [q0 to q3] is superimposed on the expansion image Im5. In addition, the puncture target region Rt is superimposed at a corresponding position on the expansion image Im5. The operator sets the puncture target region Rt via the input unit 15. The puncture support image generation unit 11 executes the superimposition of the puncture target region Rt on the expansion image Im5, for example, in the following manner.

First of all, the puncture support image generation unit 11 specifies the three-dimensional coordinates of the puncture target region Rt in the volume data. The specified three-dimensional coordinates belong to a pqr three-dimensional orthogonal coordinate system. The unit 11 then specifies the existence range of the puncture target region Rt in the polar coordinate system which defines the expansion image Im5 based on the three-dimensional coordinates of the puncture target region Rt. More specifically, the unit 11 specifies the existence range of the puncture target region Rt on the side surface of the insertion area Ro. The unit 11 then applies, to the specified existence range, a conversion expression for converting the coordinate system defining the side surface of the insertion area Ro into the polar coordinate system of the expansion image Im5, and calculates the existence range of the puncture target region Rt in the expansion image Im5. The unit 11 generates the puncture support image Im6 by superimposing a mark Mt indicating the puncture target region Rt on the existence range of the puncture target region Rt in the expansion image Im5. The mark Mt has a color that allows to visually discriminate, for example, the existence range of the puncture target region from the remaining region in the expansion image Im5. This enhances the mark Mt in the expansion image Im5to allow the operator to easily grasp the existence range of the puncture target region in the expansion image Im5. It is preferable to update the expansion image Im5 and the puncture support image Im6 every time the operator moves the distal end of the puncture needle 150. With this updating/displaying operation, as the distal end of the puncture needle 150 moves, the existence range of the puncture target region in the expansion image Im5 changes in real time. This allows the operator to grasp anatomical information in front of the puncture needle 150 in real time.

The above description has exemplified the case in which the puncture support image generation unit 11 generates a concentric distance mark with reference to a reference point. However, this embodiment is not limited to this. The unit 11 may generate a concentric distance mark with reference to the distal end portion of the puncture needle before or during insertion.

### Application Example 2

The operator keeps inserting the puncture needle to a puncture target region while observing an expansion image included in a puncture support image. It is difficult for the operator to decide the inserting direction of the puncture needle unless grasping the positional relationship between an expansion image and a real space. The puncture support image generation unit 11 according to Application Example 2 generates a puncture support image including an azimuth mark indicating the azimuth of an expansion image in a real space.

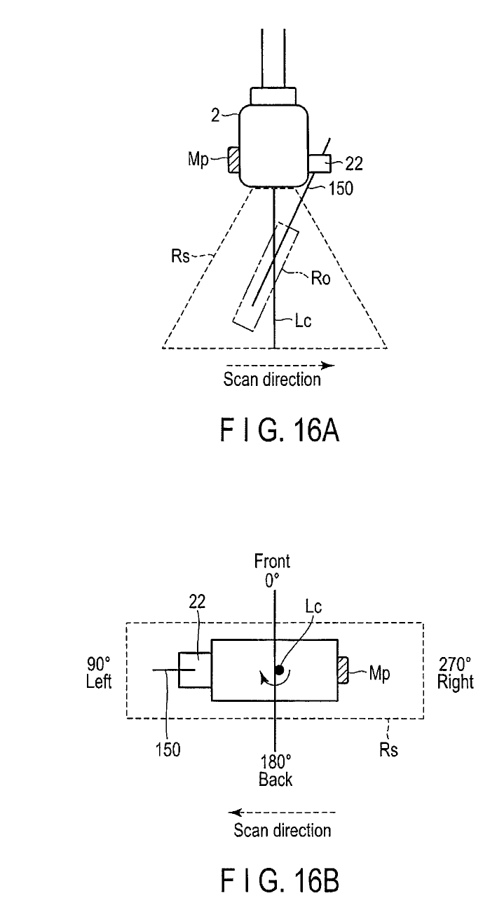

The puncture support image generation unit 11 generates an azimuth mark by using the probe mark attached to the ultrasonic probe 2. FIG. 16A is a view showing the ultrasonic probe 2 attached with a probe mark Mp when viewed from the front. FIG. 16B is a view showing the ultrasonic probe 2 attached with the probe mark Mp when viewed from above. As shown in FIGS. 16A and 16B, the ultrasonic probe 2 scans a scanning region with ultrasonic waves while sequentially transmitting/receiving ultrasonic waves along an existing scan direction. The probe mark Mp is attached to the surface of the ultrasonic probe 2. The probe mark Mp is attached to the ultrasonic probe 2 to allow the operator to grasp the scan direction of the ultrasonic probe 2. More specifically, the probe mark Mp is provided on the reference point (e.g., the start position) side in the scan direction of the surface of the housing of the ultrasonic probe 2. The puncture support image generation unit 11 stores the real space position of the probe mark Mp. For example, the real space position of the probe mark Mp is represented by the angle of the ultrasonic probe 2 around a central axis Lc. The real space position of the probe mark Mp may be expressed by an azimuth with reference to the central axis Lc of the ultrasonic probe 2. For example, in the case shown in FIGS. 16A and 16B, the real space position of the probe mark Mp is 270° or right. Note that the real space position of the probe mark Mp may be expressed by a symbol indicating north, south, east, or west or the like. The operator adjusts the direction of the ultrasonic probe 2 depending on the position of the probe mark Mp.

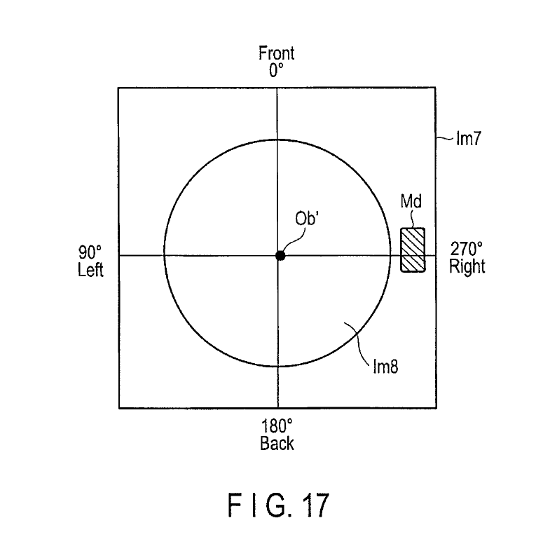

FIG. 17 shows a puncture support image Im7 including the azimuth mark Md. As shown in FIG. 17, an azimuth mark Md is superimposed on a corresponding portion around an expansion image Im8. The puncture support image generation unit 11 decides the superimposition portion of the azimuth mark Md based on the real space position of the probe mark Mp. For example, a superimposition portion is decided as follows. The unit 11 specifies the posture of an insertion area in a real space. The unit 11 specifies the posture of an insertion area in a real space based on the posture of an insertion area in volume data. The angle of the insertion area around the central axis is associated with the angle of the expansion image Im8 around the origin. The unit 11 can therefore decide the azimuth of the expansion image Im8in the real space based on the insertion area in the real space. The unit 11 specifies the placement position of a probe mark in the coordinate system of the expansion image Im8 based on the azimuth of the expansion image Im8in the real space and the real space position of the probe mark. The unit 11 superimposes the azimuth mark Md at the placement position in the puncture support image Im7. The display unit 14 displays the puncture support image Im7 on which the azimuth mark Md is superimposed. As shown in FIG. 17, the display unit 14 can display an azimuth mark indicating the azimuth of the expansion image Im8in the real space. For example, as shown in FIGS. 16A and 16B, if the real space position of the probe mark Mp is 270° (right), the azimuth mark Md is displayed on the 270° side with reference to the expansion image Im8in the puncture support image Im7.

Superimposing an azimuth mark on an expansion image allows the operator to easily understand the positional relationship between the real space and the expansion image. The operator can reliably insert the puncture needle toward a puncture target while observing an expansion image.

### Application Example 3

The inside of a patient has a complex hardness distribution due to various easy such as the types and locations of tissues. For this reason, the operator cannot sometimes linearly insert the puncture needle. An expansion image generation unit according to Application Example 3 generates an expansion image concerning hardness index values (to be referred as a hardness value expansion image hereinafter). To discriminate from a hardness value expansion image, the expansion image based on volume data in the B mode will be referred to as a B-mode expansion image. In addition, volume data in the B mode will be referred to as B-mode volume data.

It is possible to calculate hardness index values by a known method using the SWE (Shear Wave Elastography) mode. The transmission/reception unit executes ultrasonic scanning in the SWE mode. The volume data generation unit generates volume data expressing the hardness of each tissue in color (to be referred to as SWE volume data hereinafter) based on a reception signal from the reception unit. The volume data storage unit 53 stores SWE volume data. The ultrasonic diagnostic apparatus 100 may generate SWE volume data, as described above. In addition, a PACS or another ultrasonic diagnostic apparatus may transmit SWE volume data via a network.

The expansion image generation unit 9 generates a hardness value expansion image expressing a hardness index value distribution on the side surface of the insertion area Ro by using two-dimensional polar coordinates based on SWE volume data. The insertion area set in SWE volume data is identical to the insertion area set in B-mode volume data. In addition, the coordinate system of a hardness value expansion image is identical to that of a B-mode expansion image. The display unit 14 displays the hardness value expansion image. The display unit 14 may display the B-mode expansion image and the hardness value expansion image upon positional alignment and superimposition. In this case, the display unit 14 may assign a proper degree of transparency to the hardness value expansion image so as to allow visual recognition of both the hardness value expansion image and the B-mode expansion image. Observing the hardness value expansion image allows the operator to grasp the hardness distribution of the tissues in the patient. The operator can therefore insert the puncture needle 150 in consideration of the hardness of each tissue.

According to this embodiment, therefore, it is possible to improve the efficiency of puncturing operation under ultrasonic scanning.

Note that each unit included in the ultrasonic diagnostic apparatus 100 of this embodiment can be implemented by using a computer constituted by a CPU, RAM, magnetic recording device, input device, display device, and the like as hardware. For example, the system controller 16 which controls each unit of the ultrasonic diagnostic apparatus 100 can implement each type of function by causing a processor such as a CPU mounted in the computer to execute a predetermined control program. In this case, the above control program may be installed in the computer in advance. Alternatively, each control program may be stored in a computer-readable storage medium or each control program distributed via a network may be installed in the computer.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An ultrasonic diagnostic apparatus comprising:
an ultrasonic probe including a plurality of transducers;
a transmission unit configured to transmit ultrasonic waves to a scanning target region in a subject via the plurality of transducers;
a reception unit configured to receive ultrasonic waves from the scanning target region via the plurality of transducers;
a volume data generation unit configured to generate volume data concerning the scanning target region based on a reception signal from the reception unit;
a region-of-interest setting unit configured to set a predetermined range in the volume data to a region of interest, the predetermined range having a central axis coinciding with a planned insertion route of a puncture needle;
an expansion image generation unit configured to generate an expansion image expressing a brightness distribution on a side surface of the region of interest in the volume data by two-dimensional polar coordinates defined by a rotational angle around the central axis and a distance from a reference point on the central axis; and
a display unit configured to display the expansion image.

2. The ultrasonic diagnostic apparatus of claim 1, wherein the reference point includes a point included in a puncture target region, and
the region-of-interest setting unit sets the central axis of the region of interest based on position information of the puncture target region and an initial position of a distal end portion of the puncture needle.

3. The ultrasonic diagnostic apparatus of claim 1, wherein the region-of-interest setting unit sets a columnar image region to the region of interest, the columnar image region having a predetermined radius with reference to the central axis of the region of interest.

4. The ultrasonic diagnostic apparatus of claim 1, further comprising a puncture support image generation unit configured to generate a puncture support image by superimposing an indicator for supporting grasp of a position of the puncture needle in the expansion image on the expansion image upon positional alignment, and
the display unit displays the puncture support image.

5. The ultrasonic diagnostic apparatus of claim 4, wherein the puncture support image generation unit generates the puncture support image by superimposing a distance mark indicating a distance from the reference point on the expansion image upon positional alignment.

6. The ultrasonic diagnostic apparatus of claim 5, wherein the distance marks are superimposed on the expansion image at predetermined intervals from the reference point.

7. The ultrasonic diagnostic apparatus of claim 5, further comprising a detection unit configured to detect position information of the distal end of the puncture needle,
wherein the display unit displays, of the distance marks, a distance mark which corresponds to a zone through which the distal end portion of the puncture needle has passed and a distance mark corresponding to a zone through which the distal end has not passed such that the distance marks are configured to be visually discriminated from each other.

8. The ultrasonic diagnostic apparatus of claim 4, further comprising a position detection unit configured to detect position information of the distal end of the puncture needle,
wherein the puncture support image generation unit calculates an intersecting position between the puncture needle and a side surface of the region of interest based on position information of the distal end of the puncture needle and position information of the side surface of the region of interest, and generates the puncture support image by superimposing an intersecting position mark indicating the intersecting position on the expansion image upon positional alignment.

9. The ultrasonic diagnostic apparatus of claim 1, further comprising an extraction unit configured to extract pixel data concerning an anatomical region designated by a user from the volume data,
wherein the expansion image generation unit performs a coordinate conversion with respect to the pixel data and superimposes the converted pixel data on the expansion image, the coordinate conversion being the same conversion performed to a brightness distribution on the side surface of the region of interest.

10. The ultrasonic diagnostic apparatus of claim 9, wherein the anatomical region includes a luminal region.

11. The ultrasonic diagnostic apparatus of claim 1, wherein the expansion image generation unit generates another expansion image, a radial direction range of the another expansion image is limited to a predetermined range from a position of the distal end portion of the puncture needle, and
the display unit displays the another expansion image.

12. The ultrasonic diagnostic apparatus of claim 11, further comprising a puncture support image generation unit configured to generate a puncture support image by superimposing an indicator for supporting grasp of a position of the puncture needle on the another expansion image upon positional alignment, and
the display unit displays the puncture support image.

13. The ultrasonic diagnostic apparatus of claim 1, wherein the display unit displays an azimuth mark indicating an azimuth of the expansion image in a real space.

14. The ultrasonic diagnostic apparatus of claim 1, further comprising a storage unit configured to store hardness volume data expressing a spatial distribution of hardness index values in the subject,
wherein the expansion image generation unit generates another expansion image by performing a coordinate conversion with respect to a hardness index value distribution on the side surface of the region of interest in the hardness volume data, the coordinate conversion being the same conversion performed to a brightness distribution on the side surface of the region of interest, and
the display unit displays the expansion image upon superimposing the another expansion image thereon.

15. The ultrasonic diagnostic apparatus of claim 1, further comprising:
a puncture target region setting unit configured to set a puncture target region as a puncture target in accordance with an instruction from a user; and
a puncture support image generation unit configured to generate the expansion image by superimposing a mark indicating the puncture target region at a position corresponding to the puncture target region.

16. An ultrasonic image processing method comprising:
setting a predetermined range in the volume data to a region of interest, the predetermined range having a central axis coinciding with a planned insertion route of a puncture needle;
generating an expansion image expressing a brightness distribution on a side surface of the region of interest in the volume data by two-dimensional polar coordinates defined by a rotational angle around the central axis and a distance from a reference point on the central axis; and
displaying the expansion image.
